(19) United States
(12) Patent Application Publication
(10) Pub. No.: US 2003/0103947 A1
(43) Pub. Date: Jun. 5, 2003

(54) In vitro engineered cartilage constructs produced by coating biodegradable polymer with human mesenchymal stem cells

(76) Inventors: Urlich Noth, Wurzburg (DE); Rocky S. Tuan, Chester Springs, PA (US)

Correspondence Address: David S. Resnick, NIXON PEABODY LLP, 101 Federal Street, Boston, MA, 02110, US

(21) Appl. No.: 10/082,636

(22) Filed: Feb. 25, 2002

Related U.S. Application Data
(60) Provisional application No. 60/270,974, filed on Feb. 23, 2001.

(51) Int. Cl. A61K 48/00; C12N 5/08

(52) U.S. Cl. 424/93.21; 435/366

(57) ABSTRACT

The present invention discloses a population of mesenchymal stem cells (MSCs) derived from bone, more particularly, adult human trabecular bone fragments. It is demonstrated that these cells have the potential to differentiate into multiple mesenchymal lineages. Therefore, those cells may be used for treating skeletal and other connective tissue disorders.

### CONTINUING APPLICATION DATA

This application claims priority under 35 U.S.C. §119 based upon U.S. Provisional Patent Application No. 60/270,977 filed on Feb. 23, 2001.

### GOVERNMENT RIGHTS IN THE INVENTION

The invention was made in part with government support under grants AR 39740, AR 44501, AR 45181, CA 71602, DE 11327 and DE 12864 awarded by the National Institutes of Health. The government has certain rights to the invention.

### FIELD OF THE INVENTION

The present invention generally relates to the fields of cell biology and tissue engineering. More particularly, the present invention relates to a population of mesenchymal stem cells (MSCs) derived from bone, and the use of MSCs for treating skeletal and other connective tissue disorders.

### BACKGROUND OF THE INVENTION

Mesenchymal stem cells (MSCs) are cells that have the potential to differentiate into a variety of mesenchymal phenotypes by entering discrete lineage pathways. In defined culture conditions and in the presence of specific growth factors, MSCs can differentiate into cells of mesenchymal tissues such as bone, cartilage, tendon, muscle, marrow stroma, fat, dermis, and other connective tissues. The multilineage differentiation property of MSCs has opened new potential therapeutic approaches for tissue engineering.

The process for isolating and purifying MSCs from bone marrow, and in vitro mitotically expanding the population of these cells, is reported in Caplan et al. U.S. Pat. Nos. 5,197,985 and 5,226,914. However, MSCs are normally present at very low frequencies in bone marrow. In addition, the number of MSCs in bone marrow derived cells varies among individuals and decreases with age, which poses a problem of obtaining autologous MSCs for certain patients or patients in old age. Besides bone marrow, MSCs also can be obtained from other adult tissues, such as blood (including peripheral blood), periosteum, muscle, fat, and dermis. However, although MSCs derived from different tissues can differentiate into more than one mesenchymal lineage, their developmental potentials differ. Therefore, there is a need for evaluating the developmental potential of various adult cell types and identifying MSCs from various sources. The present invention meets this need by providing a new population of MSCs derived from bone.

Cultures of collagenase-treated adult human trabecular bone fragments are considered to be a reliable source of adult human osteoblastic cells (hOB) that can form a mineralized extracellular matrix in vitro, increase intracellular cAMP in response to parathyroid hormone, and express several osteoblast-related transcripts such as alkaline phosphatase (ALP), collagen type I (Col I), osteopontin (OP), osteonectin (ON), and osteocalcin (OC), which can be further elevated in response to 1α,25-dihydroxyvitamin D₃. During preparation of hOB explant cultures, collagenase pretreatment of trabecular bone fragments has been shown to effectively remove soft tissue components associated with bone surfaces, such as the periosteum and bone marrow, that may contain variable fractions of heterogeneous cells depending on the nature of the starting material (gender, donor age and site, amount of red versus yellow marrow, etc.). When these pretreated bone fragments are cultured as explants in low calcium growth medium, cells that are surrounded by mineralized matrix and protected from collagenase treatment are subsequently able to migrate from the bone fragments and begin to proliferate. While the origin of the hOB is still unclear, they have been proposed to represent osteocytes that have become liberated from their confinement and have once again become mitotic. In the past, hOBs have served as a highly useful system to study osteoblast biology, including matrix biosynthesis, cell differentiation and maturation, response to various growth factors and hormones, and cell-matrix and cell-biomaterial interactions.

The present invention, however, for the first time, demonstrates the multilineage mesenchymal differentiation potential of adult bone-derived hOB.

It is therefore, an objective of the present invention to provide a method of obtaining adult MSCs from bone.

It is a further objective of the present invention to provide a method and composition to induce regeneration of connective tissues, more particularly, cartilage and bone.

### ABBREVIATIONS

“AGN” means “aggrecan”

“ALP” means “phosphatase”

“BMP” means “bone morphogenetic proteins”

“Col I” means “collagen type I”

“Col II” means “collagen type II”

“Col IX” means “collagen type IX”

“Col X” means “collagen type X”

“DMEM” means “Dulbecco's Modified Eagle's Medium”

“FBS” means “fetal bovine serum”

“GAPDH” means “glyceraldehyde-3-phosphate dehydrogenase”

“H/E” means “haematoxylin-eosin”

“hMSC” means “human mesenchymal stem cells”

“hOB” means “human osteoblastic cells”

“LP” means “link protein”

“LPL” means “lipoprotein lipase”

“MSC” means “mesenchymal stem cells”

“OC” means “osteocalcin”

“ON” means “osteonectin”

“OP” means “osteopontin”

“PBS” means “phosphate buffered saline”

“PPARγ2” means peroxisome proliferator-activated receptor γ2

“SEM” means “scanning electron microscopy”

“TGF” means “transforming growth factor”

### DEFINITIONS

“Adipogenesis” as used herein, refers to the development of fat tissue.

“Chondrogenesis” as used herein, refers to the development of cartilage.

“Osteoblasts” as used herein, refers to bone forming cells.

“Osteogenesis” as used herein, refers to the development of bone tissue.

“Patient” as used herein, can be one of many different species, including but not limited to, mammalian, bovine, ovine, porcine, equine, rodent and human.

### BRIEF DESCRIPTION OF THE FIGURES

FIG. 1: Phase contrast photomicrographs of typical adult human trabecular bone explant cultures. (A) Appearance of adult human trabecular bone fragments after collagenase treatment. (B) hOB cells migrating from the bone fragments after approximately 10-14 days of explant culture. (C) Confluent monolayer of hOB cells after approximately 3-4 weeks of explant culture. (D) Appearance of hOB cells at the first passage. Bar=200 μm.

FIG. 2: Histological and immunohistochemical analyses of chondrogenic hOB cell pellet cultures. Left and central panel: Sections of cell pellets cultured without and with TGF-β1, respectively. Bar=200 μm. Right panel: High magnification of sections of TGF-β1-treated cell pellets. Bar=50 μm. From top to bottom: Sections were stained with haematoxylin/eosin (A-C), Alcian blue (D-F), picro-Sirius red (G-I), Col II (J-L) and link protein (M-O). Compared to untreated pellet cultures, TGF-β1-treated pellets increased substantially in size (compare size in left and central panel). The extracellular matrix of TGF-β1-treated pellets was rich in sulfated proteoglycans (D-F), birefringent fibers (G-I), specific cartilaginous matrix component such as Col II (J-L), and link protein (M-O).

FIG. 3: Histological and histochemical analysis of osteogenic and adipogenic hOB cell monolayer cultures. hOB cells were cultured without and with differentiation-stimulating supplements (left and right panel, respectively). From top to bottom: Alkaline phosphatase (A, B), Alizarin red S (C, D), and Oil red O (E, F) staining. Cell cultures treated with osteogenic supplements showed an increased number of ALP-positive cells (B) and produced a mineralized extracellular matrix (D). Treatment of hOB cells with adipogenic supplements resulted in the formation of adipocytic cells containing intracellular lipid droplets (F). In untreated cell cultures, there was an absence of such phenotypes (A, C, E). Bar=200 μm.

FIG. 4: RT-PCR analysis of the mRNA expression of lineage-specific genes in osteogenic, adipogenic, and chondrogenic cultures. Pre-confluent monolayer cultures of hOB cells migrating from trabecular bone fragments served as a control population for gene expression analysis. Under osteogenic conditions, the hOB cells expressed osteoblast-related genes (ALP, Col I, OP, OC) and AGN. The cells treated with adipogenic supplements expressed adipocyte-specific genes (LPL and PPARγ2), and also ALP and Col I. Chondrogenic cell pellet cultures treated with TGF-β1 expressed cartilage-specific genes (Col II, IX, X, AGN) and osteoblast-related Col I and OP. Untreated cells (control) expressed only Col I, consistent with a fibroblast-like phenotype. The expression of GAPDH was analyzed as a control for the RNA loading.

### DETAILED DESCRIPTION OF THE INVENTION

The present invention is directed to a population of isolated mesenchymal stem cells (MSCs), more particularly, bone-derived MSCs, and to the characterization of and uses for such cells.

MSCs are capable of differentiating into any of the specific types of mesenchymal or connective tissues, including, but not limited to, adipose, osseous, cartilaginous, elastic, and fibrous connective tissues, depending upon various influences from bioactive factors, such as cytokines.

It has been known that MSCs can be obtained from a variety of adult tissues, such as, bone marrow, blood (including peripheral blood), periosteum, muscle, fat, and dermis. There has been no report, however, of isolating MSCs from bone. In fact, cells obtained from collagenase-treated adult human trabecular bone fragments have long been considered adult human osteoblastic cells (hOB). The present invention provides a new population of MSCs derived from bone. These cells have the potential to differentiate into chondrogenic as well as osteogenic and adipogenic lineages, depending upon the culture conditions and the presence of different growth factors.

The present invention is directed to obtaining MSCs from bone fragments of any bone site of a mammal, including, but not limited to, trabecular bone and iliac crest.

Explant cultures from bone fragments may be prepared by any methods that are known to those skilled in the art. In one embodiment, the explant cultures are prepared based on a protocol first described by Robey and Termine (Robey and Termine, Calcif Tissue Int 37:453-460,1985) and modified by Sinha et al. (Sinha et al., Clin Orthop 305:258-272,1994). The cells are then plated as high-density pellet cultures (about 0.5−3×10⁵ cells/ml medium).

Employing methods similar to those described by Pittenger et al., Science 284:143-147, 1999 (incorporated herein by reference), the present invention demonstrates that high-density pellet cultures of bone-derived MSCs may differentiate into any connective tissue type in the presence of different bioactive factors. In one embodiment of the present invention, bone-derived MSCs differentiate into osteocytic cells in the presence of osteogenic supplements, including, but not limited to, ascorbate, β-glycerophosphate, BMP-2, and combinations thereof. In another embodiment of the present invention, bone-derived MSCs differentiate into adipocytic cells in the presence of adipogenic supplements, including, but not limited to, dexamethasone, IBMS, insulin, indomethacin and combinations thereof. In yet another embodiment of the present invention, bone-derived MSCs developed chondrocytic characteristics in the presence of members of the transforming growth factor-β superfamily.

Bone marrow-derived MSCs have been used in tissue engineering. For example, the following are a number of U.S. patents that are directed to this matter.

U.S. Pat. No. 5,197,985 to Caplan et al. describes a method of treating connective tissue disorders by providing culturally expanded purified marrow-derived mesenchymal cells, and applying the culturally expanded purified marrow-derived mesenchymal cells to a desired area of connective tissue regeneration, such as an area of connective tissue damage, by means of a vehicle or carrier, more particularly, a porous ceramic composition comprised of tri-calcium phosphate or hydroxyapatite or combinations of the two, under conditions suitable for differentiating the cells present in the carrier into the type of connective tissue desired, such as the type of connective tissue necessary for repair.

U.S. Pat. No. 5,226,914 to Caplan et al. describes a method for enhancing the implantation of a prosthetic device into skeletal tissue. The method comprises the steps of providing culturally expanded purified marrow-derived mesenchymal cells, adhering the culturally expanded mesenchymal cells onto the connective surface of a prosthetic device, and implanting the prosthetic device containing the culturally expanded purified marrow-derived mesenchymal cells under conditions suitable for differentiating the cells into the type of skeletal or connective tissue needed for implantation.

U.S. Pat. No. 6,214,369 to Grande et al. describes that mesenchymal stem cells (MSCs) in a polymeric carrier implanted into a cartilage and/or bone defect will differentiate to form cartilage and/or bone, as appropriate. Suitable polymeric carriers include porous meshes or sponges formed of synthetic or natural polymers, as well as polymer solutions.

(The disclosures of the above-referenced patents and publications are incorporated herein by reference.)

It is within the scope of the present invention to use bone-derived MSCs, bone derived MSCs in combination with a pharmaceutical acceptable carrier(s), or bone-derived MSCs in combination with a pharmaceutical acceptable bioactive factor(s) for treating skeletal and other connective tissue disorders. Suitable carriers include, but are not limited to, collagen, hyaluronan, gelatin, alginate gels, demineralized bone matrix (DBM), biodegradable polymers, calcium-phosphates and hydroxyapatite.

It is still within the scope of the present invention, that bone-derived MSCs are genetically engineered as an effective cellular vehicle to deliver gene products, such as those members of the TGF-β superfamily. Techniques of introducing foreign nucleic acid, e.g. DNA, encoding certain gene products are well known in the arts. Those techniques include, but are not limited to, calcium-phosphate-mediated transfection, DEAE-mediated transfection, microinjection, retroviral transformation, protoplast fusion, and lipofection. The genetically-engineered MSCs may express the foreign nucleic acid transiently or stably. In general, transient expression occurs when the foreign DNA does not stably integrate into the chromosomal DNA of the transfected MSC. In contrast, long-term expression of foreign DNA occurs when the foreign DNA has been stably integrated into the chromosomal DNA of the transfected MSC.

Methods

Preparation of Collagenase-Treated Trabecular Bone Explants Cultures

All chemicals were purchased from Sigma Chemicals (St. Louis, Mo., U.S.A.) unless otherwise stated. Trabecular bone fragments were obtained from the femoral head of patients (2 females aged 42 and 58 years, and 2 males aged 47 and 54 years) undergoing total hip arthroplasty. None of the patients had a history of osteoporosis or avascular necrosis. Explant cultures were prepared based on a protocol first described by Robey and Termine (Robey and Termine, Calcif Tissue Int 37:453-460,1985) and modified by Sinha et al.(Sinha et al., Clin Orthop 305:258-272,1994). Trabecular bone fragments were harvested using a bone curet, transferred to glass vials containing DMEM/F-12K (Speciality Media, Phillipsburg, N.J., U.S.A.) supplemented with antibiotics (50 I.U. penicillin/ml, 50 μg streptomycin/ml, Celigro, Herndon, Va., U.S.A.), minced extensively with surgical scissors, and washed repeatedly with DMEM/F-12K. Bone fragments were next transferred to a spinner flask containing DMEM/F-12K supplemented with 2 mM L-glutamine, 50 μg/ml ascorbate, 256 U/ml collagenase type XI and antibiotics, and incubated at 37° C. for 3-4 h in a humidified 95% air—5% CO₂ atmosphere until the cellular material on the bone surface disappeared, as assessed by light microscopy. Following extensive rinsing with 0.9% sodium chloride (Baxter, Deerfield, Ill., U.S.A.), bone fragments were plated in a tissue culture flask containing calcium-free DMEM/F12-K supplemented with 10% fetal bovine serum (FBS, Premium Select, Atlanta Biologicals, Atlanta, Ga., U.S.A.), 2 mM L-glutamine, 50 μg/ml ascorbate, and antibiotics. Explant cultures were maintained at 37° C. in a humidified 95% air—5% CO₂ atmosphere with the medium changed every 3-4 days. When the cells growing out of the explants reached 70-80% confluence (after approximately 3-4 weeks), they were detached from the bottom of the tissue culture flasks with 0.25% trypsin containing 1 mM EDTA (Gibco BRL, Life Technologies, Grand Island, N.Y., U.S.A.), counted in a hemocytometer, and plated as high-density pellet cultures or monolayers.

Chondrogenic Differentiation of High-Density Pellet Cultures

For chondrogenic differentiation, cells were plated as high-density pellet cultures in a chemically defined, serum-free DMEM (BioWhittaker, Walkersville, Md., U.S.A.) as described previously in Johnstone et al., Exp Cell Res 238:265-272, 1998; Mackay et al., Tissue Eng 4:415-428, 1998; Pittenger et al., Science 284:143-147, 1999; and Yoo et al., J Bone Joint Surg 80:1745-1757, 1998. Aliquots of 2×10⁵ cells in 0.5 ml medium were pelleted by centrifugation at 500× g for 5 min in 15-ml conical polypropylene tubes, and the resulting cell pellets were supplemented with 10 ng/ml transforming growth factor-β1 (TGF-β1; R&D, Minneapolis, Minn., U.S.A) to stimulate chondrogenic differentiation of the cells. Control cultures were maintained in a chemically defined, serum-free medium without TGF-β1. High-density pellet cultures were maintained for 3 weeks at 37° C. in a humidified 95% air—5% CO₂ atmosphere. The medium was changed every 3-4 days with TGF-β1 added fresh to the appropriate culture.

Osteogenic and Adipogenic Differentiation of Monolayer Cultures

For osteogenic and adipogenic differentiation, cells at the density of 1.5×10⁵ cells/ml DMEM/F-12K (osteogenic differentiation) or DMEM (adipogenic differentiation) supplemented with 10% FBS and antibiotics, were plated in 2-well chamber slides (Nalge Nunc, Naperville, Ill., U.S.A.) and grown to confluence. Osteogenic differentiation of confluent monolayer cultures was then induced with 50 μg/ml ascorbate, 10 mM β-glycerophosphate, and 30 ng/ml human recombinant bone morphogenetic protein-2 (BMP-2; kindly provided by Genetics Institute, Cambridge, Mass., U.S.A.) (Lecanda et al., J Cell Biochem 67:386-398, 1997), whereas adipogenic differentiation was induced with 1 μM dexamethasone, 0.5 mM 3-isobutyl-1-methylxanthine (IBMX), 1 μg/ml insulin and 100 μM indomethacin (Pittenger et al., Science 284:143-147,1999). Control cultures were grown without osteogenic or adipogenic supplements. Osteogenic and adipogenic stimulation was carried out for 4 and 2 weeks, respectively, with the media changed every 3-4 days and supplements added fresh to each culture.

Histological Histochemical and Immunohistochemical Analysis

Chondrogenic high-density pellet cultures were rinsed with phosphate buffered saline (PBS), fixed in 2% paraformaldehyde, dehydrated in ethanol, infiltrated with isoamyl alcohol, and embedded in paraffin. Sections of 8 μm thickness were obtained through the center of each pellet and mounted on microscope slides. The sections were then stained with haematoxylin-eosin, Alcian blue, or picro-Sirius red as described previously (Denker et al., Differentiation 64:67-76, 1999; Dharmavaram et al., Arthritis Rheum 7:1433-1442, 1999; and Haas et al., Differentiation 64:77-89, 1999). For collagen type II (Col II) or link protein (LP) detection, sections were pre-digested for 15 min at 37° C. with 300 U/ml hyaluronidase or 1.5 U/ml chondroitinase ABC, respectively. Sections were then incubated with the monoclonal antibodies, II-II6B3 (15 μg/ml PBS) specific to Col II or 8-A-4 (6 μg/ml PBS) specific to LP (Developmental Studies Hybridoma Bank Iowa City, Iowa, U.S.A.), for 1 h at 37° C. or overnight at 4° C., respectively. Immunostaining was detected calorimetrically using Histostain-SP Kit for DAB (Zymed Laboratories Inc., San Francisco, Calif., U.S.A.). Osteogenic monolayer cultures were stained histochemically for ALP (Sigma Cat. No. 86-C) according to the manufacturer 's protocol and for matrix mineralization using Alizarin red S as described previously (Bodine et al., J Bone Miner Res 11:806-819,1996). Adipogenic monolayer cultures were stained histochemically for intracellular lipid droplets with Oil red O as described previously (Pittenger et al., Science 284:143-147, 1999).

RNA Isolation and RT-PCR Analysis of Gene Expression

Total cellular RNA was extracted with Trizol reagent (Gibco BRL, Life Technologies, Grand Island, N.Y., U.S.A.). For efficient RNA extraction from high-density pellet cultures, pellets were first briefly homogenized in Trizol reagent. The isolated RNA samples were converted to cDNA using random hexamers and Superscript II RNase H-Reverse Transcriptase (SuperScript First-Strand Synthesis System, Gibco BRL, Life Technologies, Grand Island, N.Y., U.S.A.), and then amplified by PCR using AmpliTaq DNA Polymerase (Perkin Elmer, Norwalk, Conn., U.S.A.) and the gene-specific primer sets listed in Table 1. Expression of the following genes was examined: collagen type I (Col IA2), alkaline phosphatase (ALP), osteopontin (OP), osteocalcin (OC), LPL (lipoprotein lipase), peroxisome proliferator-activated receptor γ2 (PPAR γ2), collagen type II (Col II), collagen type IX (Col IX), collagen type X (Col X), and aggrecan (AGN). Amplifications were performed for 34 (OC) or 32 (all other genes) cycles consisting of 1-min denaturation at 95° C., 1-min annealing at 60° C. (OC), 57° C. (Col II, IX, X, AGN), or 51° C. (all other genes) and 1-min extension at 72° C., with the initial denaturation at 95° C. for 1 min and final incubation at 72° C. for 10 min. In all RT-PCR assays, the housekeeping gene glyceraldehyde-3-phosphate dehydrogenase (GAPDH) was analyzed to monitor RNA loading. RT-PCR products were analyzed by electrophoresis in 2% MetaPhor agarose gel (FMC Corp., Rockland, Me., U.S.A.) containing ethidium bromide.

TABLE 1
PCR primer sets for amplification of lineage-specific genes and
the length of amplified products

| | | | |
|---|---|---|---|
| | Primer sequence: | Product | |
| Gene | sense/antisense (5′-3′) | size | Reference |

Osteogenic markers

| | | | | |
|---|---|---|---|---|
| CollA2 | GGACACAATGGATTGCAAGG | (SEQ. NO.1) | 461 bp | Lomri et al., |
| | TAACCACTGCTCCACTCTGG | (SEQ. NO.2) | | Calcif Tissue |
| | | | | Int. 64:394-401, |
| | | | | 1999 |
| ALP | TGGAGCTTCAGAAGCTCAACACCA | (SEQ. NO.3) | 453 bp | Pittenger et |
| | ATCTCGTTGTCTGAGTACCAGTCC | (SEQ. NO.4) | | al., Science |
| | | | | 284: 143-147, |
| | | | | 1999 |
| OP | ACGCCGACCAAGGAAAACTC | (SEQ. NO.5) | 483 bp | Gene Bank |
| | GTCCATAAACCACACTATCACCTCG | (SEQ. NO.6) | | Access No. BC |
| | | | | 007016 |
| OC | ATGAGAGCCCTCACACTCCTC | (SEQ. NO.7) | 297 bp | Lomri et al., |
| | GCCGTAGAAGCGCCGATAGGC | (SEQ. NO.8) | | Calcif Tissue |
| | | | | Int 64:394-401, |
| | | | | 1999 |

Adipogenic markers

| | | | | |
|---|---|---|---|---|
| LPL | GAGATTTCTCTGTATGGCACC | (SEQ. NO.9) | 276 bp | Rickard et al., |
| | CTGCAAATGAGACACTTTCTC | (SEQ. NO.10) | | J Bone Miner |
| | | | | Res. 11:312-324, |
| | | | | 1996 |
| PPARγ2 | GCTGTTATGGGTGAAACTCTG | (SEQ. NO.11) | 352 bp | Pittenger et al., |
| | ATAAAGGTGGAGATGCAGGCTC | (SEQ. NO.12) | | Science 284:143-147 |
| | | | | 1999 |

Chondrogenic markers

| | | | | |
|---|---|---|---|---|
| Col | TTTCCCAGGTCAAGATGGTC | (SEQ. NO.13) | 377 bp | Pittenger et |
| II | CTTCAGCACCTGTCTCACCA | (SEQ. NO.14) | | al., Science |
| | | | | 284:143-147 |
| | | | | 1999 |
| Col IX | GGGAAAATGAAGACCTGCTGG | (SEQ. NO.15) | 516 bp | Gene Bank |
| | CCAAAAGGCTGCTGTTTGGAGAC | (SEQ. NO.16) | | Access No. NM |
| | | | | 001851 |
| Col X | GCCCAAGAGGTGCCCCTGGAATAC | (SEQ. NO.17) | 703 bp | Johnstone et al., |
| | CCTGAGAAAGAGGAGTGGACATAC | (SEQ. NO.18) | | Exp Cell Res |
| | | | | 238:265-272, |
| | | | | 1998 |
| AGN | TGAGGAGGGCTGGAACAAGTACC | (SEQ. NO.19) | 350 bp | Gene Bank |
| | GGAGGTGGTAATTGCAGGGAACA | (SEQ. NO.20) | | Access No. NM |
| | | | | 001135 |

Internal control

| | | | | |
|---|---|---|---|---|
| GAPDH | GGGCTGCTTTTAACTCTGGT | (SEQ. NO.21) | 702 bp | Lomri et al., Calcif |
| | TGGCAGGTTTTTCTAGACGG | (SEQ. NO.22) | | Tissue Int 64:394-401, |
| | | | | 1999 |

Results

Morphological Observation of Collagenase-Treated Trabecular Bone Explant Cultures

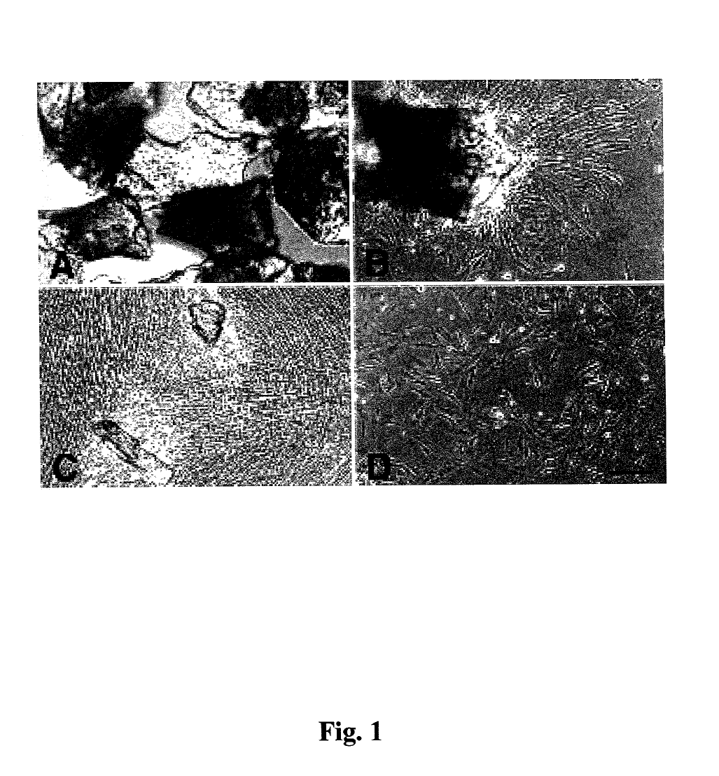

After 3-4 hours of collagenase treatment the surface of the bone fragments appeared devoid of cellular material and soft tissue components as observed by light microscopy (FIG. 1A). When these bone fragments were plated in low calcium DMEM/F-12K medium, cells appeared migrating from the explants after approximately 10-14 days (FIG. 1B). With continued incubation in low calcium DMEM/F-12K medium, the cells proliferated and formed a confluent monolayer after approximately 21-28 days (FIG. 1C). The cells appeared as a homogeneous fibroblastic cell population with mitotic figures (FIG. 1D). No differences in growth characteristics or cell morphology were noted among the different patient samples.

Histoloqical and Immunohistochemical Examination of Chondrogenic Cultures

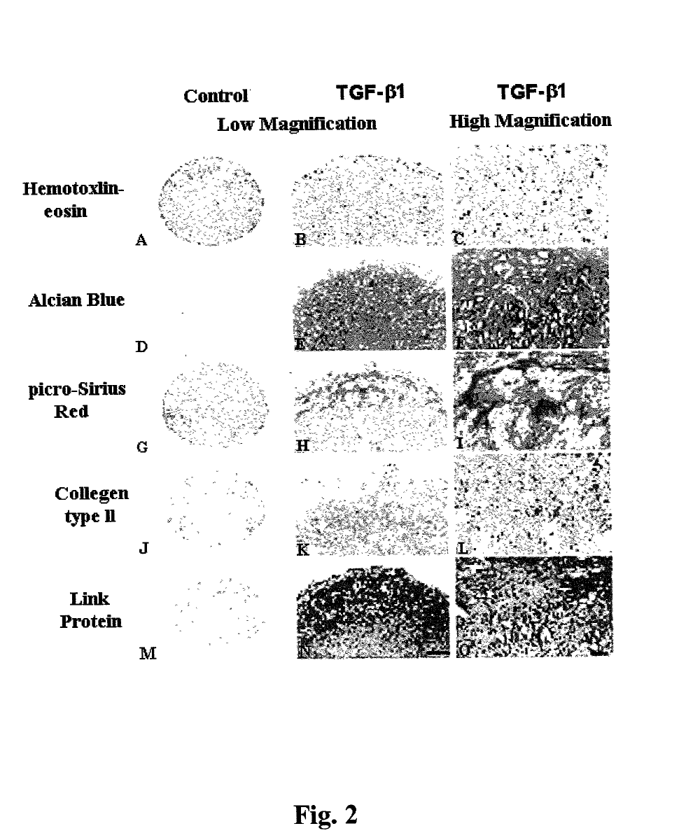

All high-density pellet cultures, formed by centrifugation, detached spontaneously from the bottom of polypropylene conical tubes within 24 h and were further cultured in suspension in a chemically-defined, serum-free medium with or without TGF-β1. Over the 3-week culture period, pellet cultures treated with TGF-β1 increased in size, while omission of TGF-β1 prevented any size increase of the pellets (compare size in FIG. 2). Haematoxylin-eosin stained sections of 3-week TGF-β1-treated pellets showed morphologically distinct, chondrocyte-like cells embedded in abundant extracellular matrix (FIGS. 2B, C). Alcian blue staining of these sections revealed the presence of a sulfated, proteoglycan-rich extracellular matrix (FIGS. 2E, F), while picro-Sirius red staining showed prominent birefringent fibers present in the matrix and surrounding the cells (FIGS. 2H, I). Cells within untreated pellets did not display chondrocyte-like morphology (FIG. 2A) or elaborate a proteoglycan-rich extracellular matrix (FIG. 2D), and no significant birefringent fibers in the matrix were detected (FIG. 2G). Also, only sections of TGF-β1-treated pellets immunostained for Col II (FIGS. 2K, L) and LP (FIGS. 2N, O) in the extracellular matrix, while neither Col II nor LP were detected in sections of untreated pellets (FIGS. 2J, M). The cells from all tested donors responded similarly during chondrogenic high-density pellet cultures.

Histological and Histochemical Examination of Osteogenic and Adipogenic Cultures

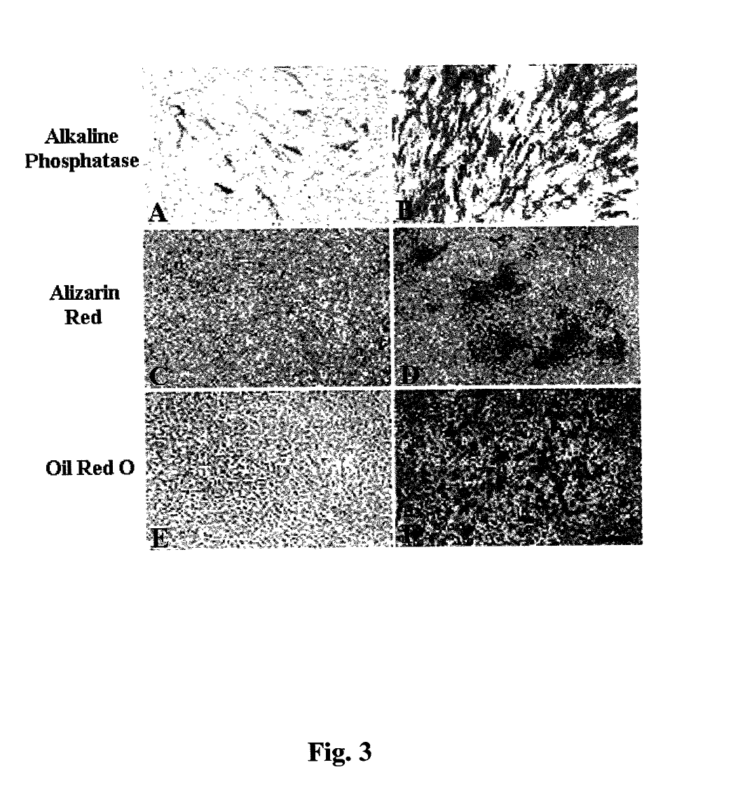

Confluent monolayer cultures treated for 10 days with the osteogenic supplements, ascorbate, β-glycerophosphate and BMP-2, showed a marked increase of ALP-positive cells (FIG. 3B) as compared to control cultures grown without osteogenic supplements (FIG. 3A). In cultures maintained for longer times, cells treated with osteogenic supplements began to produce mineralized matrix as observed by phase contrast microscopy and further confirmed by Alizarin red S staining (4-week treated cultures, FIG. 3D), while control cultures did not mineralize (FIG. 3C). Confluent monolayer cultures treated with the adipogenic supplements—dexamethasone, IBMX, insulin and indomethacin—showed the first adipocytic cells containing intracellular lipid droplets as early as treatment day 3 as observed by phase contrast microscopy and further confirmed by Oil red O staining (2-week treated cultures, FIG. 3F). Control cultures grown without adipogenic supplements showed no formation of adipocytic cells containing intracellular lipid droplets (FIG. 3E). The cells from all tested donors responded similarly in osteogenic and adipogenic culture conditions.

Expression of Lineage-Specific Genes in Chondrogenic, Osteogenic and Adipogenic Cultures

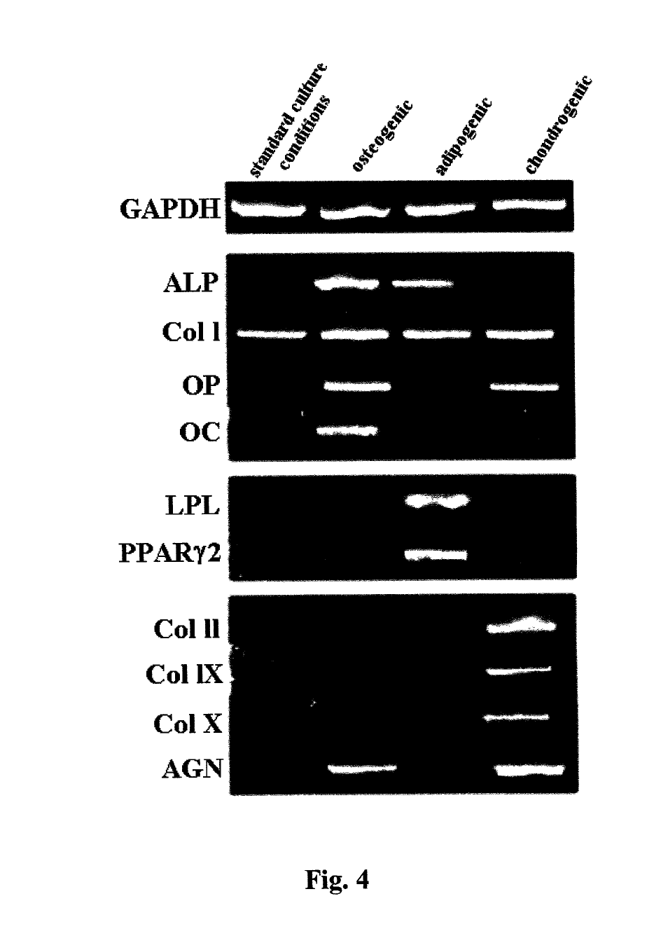

Pre-confluent monolayer cultures of cells migrating from trabecular bone fragments served as a control population for gene expression analysis. These primary cells cultured without differentiation-stimulating agents showed the expression of Col I mRNA but not other osteoblast-related genes such as ALP, OP, and OC. Expression of the adipocyte-specific genes, LPL and PPARγ2, or the chondrocyte-associated genes, Col II, Col IX, Col X, and AGN, also was not detected (FIG. 4, control). In contrast, cells cultured as monolayers and treated for 3 weeks with osteogenic supplements expressed ALP, Col I, OP and OC genes, indicating osteogenic differentiation. Interestingly, these cells also expressed the AGN gene but did not express other chondrocyte-associated or adipocyte-specific genes (FIG. 4, osteogenic). On the other hand, cells cultured as monolayers and treated for 2 weeks with adipogenic supplements expressed LPL and PPARγ2 genes, indicative of adipogenic differentiation. These cells also expressed osteoblast-related genes, ALP, Col I and OP, but not OC or chondrocyte-associated genes (FIG. 4, adipogenic). Finally, cells grown as chondrogenic high-density pellet cultures for 3 weeks in chemically-defined, serum-free medium supplemented with TGF-β1 expressed the chondrocyte-associated genes: Col II, IX, X and AGN. These cells also showed expression of Col I and OP genes, but not ALP or OC, or the adipocyte-specific genes, LPL and PPARγ2 (FIG. 4, chondrogenic). The gene expression pattern in control and differentiation-stimulating conditions was identical for all tested donor cell populations.

Discussion

The present invention investigated the developmental potential of cells derived from adult human femoral trabecular bone, namely the cells ability to differentiate in vitro into cell types representative of chondrogenic, osteogenic and adipogenic lineages. The results show that cells derived from collagenase-treated trabecular bone fragments differentiate in vitro into these three examined mesenchymal lineages when cultured in defined conditions similar to those previously described for adult human bone marrow-derived mesenchymal stem cells (hMSC) (Pittenger et al., Science 284:143-147, 1999).

A number of cell culture models are currently in use for the study of adult human primary osteoblasts, including osteoblast precursor cells originating from bone marrow, cells of the osteoblast lineage derived from explants of adult human trabecular bone, and collagenase-pretreated trabecular bone fragments. The last method has been claimed to yield a more homogenous osteoblastic cell population based on the observation that collagenase digestion of trabecular bone fragments efficiently removes connective tissue components so that cell populations are eventually derived only from those cells within the osteoid matrix. When these collagenase-pretreated trabecular bone fragments were further plated in a low calcium medium to facilitate matrix dissolution, it was observed that cell proliferation was initially evident in close proximity to the surfaces of the explants and only after approximately two weeks of culture.

Interestingly, the predominant cell type in the explant cultures disclosed in the present invention have an elongated, fibroblast-like morphology and, in both initial and post-confluent cultures, do not spontaneously acquire a more polygonal morphology, which is considered by some investigators to reflect a more “mature” osteoblast-like phenotype. RT-PCR analysis of pre-confluent hOB cells that had just migrated from the bone fragments showed that these cells express Col I, but not other osteoblast-related genes. Moreover, in further differentiation assays carried out on first passage confluent monolayers of hOB cells, control cultures stained weakly for ALP, the most-widely used biochemical marker of osteoblasts. These results imply that the cells that migrate from collagenase-pretreated trabecular bone fragments when cultured in standard culture conditions display an undifferentiated and/or dedifferentiated cell phenotype.

To assess the in vitro developmental potential of hOB, the present invention uses a similar approach to that previously described for adult human mesenchymal stem cells (hMSCs) (Pittenger et al., Science 284:143-147, 1999). The multilineage differentiation potential of adult hMSCs has been well established. These cells, when cultured as high-density pellet cultures in a serum-free, chemically-defined medium containing dexamethasone, ascorbate, sodium pyruvate, proline and TGF-β1, have been shown to develop a chondrocyte-like phenotype. This observation has opened the possibility of using these cells for the reconstruction of cartilage defects in tissue engineering. The in vitro osteogenic and adipogenic differentiation abilities of hMSCs also have been well documented (Bruder et al., J Cell Biochem 56:283-94,1994; Jaiswal et al., J Cell Biochem 64:295-312, 1997; Minguell et al., Exp Biol Med 226:507-520,2001; and Pittenger et al., Science 284:143-147, 1999). However, there is a growing body of evidence implying that not only marrow stroma-derived cells, but also more defined cell types of mesenchymal origin, such as adipocytes, myoblasts and chondrocytes, can differentiate or transdifferentiate into other cell types in addition to their default lineage. Recently, periosteally derived cells, which attain an osteoblast-like phenotype in culture, have been shown to differentiate into chondrocytes when further cultured in suspension in agarose gels (Bahrami et al., Anat Rec 259:124-30, 2000).

The present invention discloses that chondrogenic differentiation of hOB can be achieved in high-density pellet cultures in a serum-free, chemically defined medium containing TGF-β1. The size of TGF-β1 treated pellets increased over the 3-week culture period and, as previously shown for adult hMSC (Johnstone et al., Exp Cell Res 238:265-272,1998; Mackay et al., Tissue Eng 4:415-428,1998; and Yoo et al., J Bone Joint Surg 80:1745-1757,1998), this effect appeared almost entirely due to the deposition of extracellular matrix rather than to continued cell division, as evidenced by histochemical and immunohistochemical analysis. Furthermore, RT-PCR analysis revealed the expression of Col II, IX, X and AGN transcripts, characteristic of the chondrocyte phenotype. It is noteworthy that the expression of Col X was upregulated in the TGF-β1 treated hOB pellet cultures. The significance of Col X transcription at the early phase of chondrogenic differentiation is unclear, since Col X is generally considered a component of mature hypertrophic cartilage. This may indicate that at this stage of culture, the hOB cells are in a transitional state, expressing transcripts characteristic of both osteoblastic and chondrocytic lineages. It is noteworthy that Yoo et al (1998) also detected by immunostaining, as early as culture Day 5, Col X associated with the cell surface of hMSC maintained under similar chondrogenic conditions.

In monolayer culture, osteoblastic differentiation involves a programmed developmental sequence, which is characterized by an early proliferative stage, followed by extracellular matrix development and maturation, and matrix mineralization. During this process, ALP expression and activity progressively increase, then decrease when mineralization progresses. The cells also upregulate expression of several osteoblast-related genes such as Col I, OP and OC. In the present invention, hOB cultured in the presence of osteogenic supplements, ascorbate, β-glycerophosphate and BMP-2, showed an increased number of ALP-positive cells, expressed ALP, Col I, OP and OC transcripts and formed a mineralized matrix, all characteristic of the osteoblastic phenotype. Although many cell culture models employ dexamethasone as an osteo-inductive agent, the usage of BMP-2 is more appropriate, since the osteo-inductive effect of BMP-2, in contrast to glucocorticoids, can be achieved both in vitro and in vivo. The osteo-inductive effect of BMP-2 on human osteoblasts and human bone marrow stromal cell cultures has been reported (Lecanda et al., J Cell Biochem 67:386-398,1997). Notably, hOB cells in the osteogenic cultures, as disclosed in the present invention, also expressed AGN, a proteoglycan core protein expressed predominantly in cartilaginous tissues. The role of AGN in osteoblastic differentiation has not been investigated, although its expression has been found at low levels in ROS17/2.8 osteosarcoma cells and in intramembranous bone of the chick embryo. Perhaps AGN functions as other small proteoglycans, such as decorin, in the mineralization process by binding to and regulating the fibril length of collagen. That expression of decorin is selectively stimulated by BMP-2 in human osteoblasts and human bone marrow stromal cell cultures implies, although indirectly, a similar mechanism for BMP-2 action on AGN gene expression in the culture system of the instant invention.

Furthermore, the results also show that treatment of hOB monolayer cultures for 2 weeks with the adipogenic supplements, dexamethasone, IBMX, insulin and indomethacin, result in the hOB cells conversion to adipocytes, as evidenced by the appearance of cells containing intracellular lipid droplets and gene expression of LPL and PPARγ2. These results are consistent with the known characteristics of the adipogenic differentiation pathway, i.e., that it is not only accompanied by changes in cellular morphology and the formation of cytoplasmic lipid droplets but also by transcriptional activation of many genes.

Interestingly, the hOB cultures of the instant invention when treated with adipogenic supplements also showed ALP gene expression. However, adipocytes have been shown to express ALP (Beresford et al., Metab Bone Dis Rel Res 5:229-34,1984; Beresford et al., Am J Med Genet 45:163-178,1993; Dorheim et al., J Cell Physiol 154:317-328,1993; and Okochi et al., Clin Chim Acta 162:19-27,1987). Alternatively, only approximately 30-40% of the hOB cells in the adipogenic cultures become adipocytes, as evidenced by Oil red O staining of cytoplasmic lipid droplets, therefore, the Oil red O negative cells could account for the detection of ALP by RT-PCR.

The results demonstrate that the cells derived from collagenase-pretreated adult human trabecular bone fragments display mesenchymal progenitor characteristics. The finding that the cells derived from human trabecular bone fragments, traditionally considered as osteoblastic cells, are able to develop into three distict mesenchymal cell phenotypes under controlled in vitro culture conditions, raise interesting questions on the developmental plasticity of cells normally residing within the mineralized matrix of mature bone.

While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. An isolated, homogeneous population of mesenchymal stem cells which can differentiate into cells of more than one connective tissue type, wherein said mesenchymal stem cells are obtained from bone.

2. The mesenchymal stem cells of claim 1, wherein said mesenchymal stem cells are obtained from human trabecular bone.

3. The mesenchymal stem cells of claim 1, wherein said mesenchymal stem cells are obtained from human iliac crest.

4. The mesenchymal stem cells of claim 1, wherein one of said connective tissue types is selected from the group consisting of bone, cartilage, adipose, tendon, ligament, and dermis.

5. The mesenchymal stem cells of claim 1, wherein said mesenchymal stem cells are transiently or stably genetically engineered to express one or more gene products.

6. The mesenchymal stem cells of claim 5, wherein said one or more gene products are members of the transforming growth factor-β superfamily.

7. A therapeutic composition comprising the mesenchymal stem cells of claim 1 and a pharmaceutically acceptable carrier, wherein said mesenchymal stem cells are present in an amount effective to produce connective tissue cells.

8. The therapeutic composition of claim 7, wherein said connective tissue is selected from the group consisting of bone, cartilage, adipose, tendon, ligament, and dermis.

9. The therapeutic composition of claim 7, wherein said mesenchymal stem cells are transiently or stably genetically engineered to express one or more gene products.


SEQUENCE LISTING (22 sequences)

SEQ ID NO: 1 - DNA, 20 bp, Artificial Sequence
  synthetic oligonucleotide primer
ggacacaatggattgcaagg

SEQ ID NO: 2 - DNA, 20 bp, Artificial Sequence
  synthetic oligonucleotide primer
taaccactgctccactctgg

SEQ ID NO: 3 - DNA, 24 bp, Artificial Sequence
  synthetic oligonucleotide primer
tggagcttcagaagctcaacacca

SEQ ID NO: 4 - DNA, 24 bp, Artificial Sequence
  synthetic oligonucleotide primer
atctcgttgtctgagtaccagtcc

SEQ ID NO: 5 - DNA, 20 bp, Artificial Sequence
  synthetic oligonucleotide primer
acgccgaccaaggaaaactc

SEQ ID NO: 6 - DNA, 25 bp, Artificial Sequence
  synthetic oligonucleotide primer
gtccataaaccacactatcacctcg

SEQ ID NO: 7 - DNA, 21 bp, Artificial Sequence
  synthetic oligonucleotide primer
atgagagccctcacactcctc

SEQ ID NO: 8 - DNA, 21 bp, Artificial Sequence
  synthetic oligonucleotide primer
gccgtagaagcgccgataggc

SEQ ID NO: 9 - DNA, 21 bp, Artificial Sequence
  synthetic oligonucleotide primer
gagatttctctgtatggcacc

SEQ ID NO: 10 - DNA, 21 bp, Artificial Sequence
  synthetic oligonucleotide primer
ctgcaaatgagacactttctc

SEQ ID NO: 11 - DNA, 21 bp, Artificial Sequence
  synthetic oligonucleotide primer
gctgttatgggtgaaactctg

SEQ ID NO: 12 - DNA, 22 bp, Artificial Sequence
  synthetic oligonucleotide primer
ataaaggtggagatgcaggctc

SEQ ID NO: 13 - DNA, 20 bp, Artificial Sequence
  synthetic oligonucleotide primer
tttcccaggtcaagatggtc

SEQ ID NO: 14 - DNA, 20 bp, Artificial Sequence
  synthetic oligonucleotide primer
cttcagcacctgtctcacca

SEQ ID NO: 15 - DNA, 21 bp, Artificial Sequence
  synthetic oligonucleotide primer
gggaaaatgaagacctgctgg

SEQ ID NO: 16 - DNA, 23 bp, Artificial Sequence
  synthetic oligonucleotide primer
cgaaaaggctgctgtttggagac

SEQ ID NO: 17 - DNA, 24 bp, Artificial Sequence
  synthetic oligonucleotide primer
gcccaagaggtgcccctggaatac

SEQ ID NO: 18 - DNA, 24 bp, Artificial Sequence
  synthetic oligonucleotide primer
cctgagaaagaggagtggacatac

SEQ ID NO: 19 - DNA, 23 bp, Artificial Sequence
  synthetic oligonucleotide primer
tgaggagggctggaacaagtacc

SEQ ID NO: 20 - DNA, 23 bp, Artificial Sequence
  synthetic oligonucleotide primer
ggaggtggtaattgcagggaaca

SEQ ID NO: 21 - DNA, 20 bp, Artificial Sequence
  synthetic oligonucleotide primer
gggctgcttttaactctggt

SEQ ID NO: 22 - DNA, 20 bp, Artificial Sequence
  synthetic oligonucleotide primer
tggcaggtttttctagacgg